(19) United States
(12) Patent Application Publication
(10) Pub. No.: US 2026/0364124 A1
(43) Pub. Date: Nov. 19, 2026

(54) ANTI-C. DIFFICILE SMALL MOLECULE METABOLITES FROM DIET AND GUT MICROBIOME

(71) Applicant: The Children's Medical Center Corporation, Boston, MA (US)

(72) Inventors: Seth Rakoff-Nahoum, Boston, MA (US); Gavin A. Kuziel, Boston, MA (US)

(73) Assignee: The Children's Medical Center Corporation, Boston, MA (US)

(21) Appl. No.: 19/486,658

(22) PCT Filed: May 22, 2024
(86) PCT No.: PCT/US2024/030456

§ 371 Date: Nov. 21, 2025

Related U.S. Application Data
(60) Provisional application No. 63/503,914, filed on May 23, 2023.

(51) Int. Cl. A61K 35/74; A61K 31/05; A61K 31/12; A61K 31/7034; A61P 31/04

(52) U.S. Cl. CPC A61K 35/74; A61K 31/05; A61K 31/12; A61K 31/7034; A61P 31/04

(57) ABSTRACT

The present disclosure provides pharmaceutical compositions comprising a microorganism capable of expressing an enzyme that converts a stilbene glycoside (e.g., polydatin) or a dihydrochalcone glycoside (e.g., phloridzin) to a stilbene (e.g., resveratrol) or a dihydrochalcone (e.g., phloretin), and a stilbene glycoside or a dihydrochalcone glycoside, or a pharmaceutically acceptable salt thereof. The present disclosure also provides methods for treating a Clostridioid. es difficile infection in a subject in need thereof comprising administering to the subject a therapeutically effective amount of any of the pharmaceutical compositions described herein. Methods for treating a Clostridioides difficile infection in a subject in need thereof comprising administering a therapeutically effective amount of a pharmaceutical composition comprising a stilbene glycoside, a dihydrochalcone glycoside, a stilbene, or a dihydrochalcone, or a pharmaceutically acceptable salt thereof, are also provided herein. The present disclosure further provides kits, food products, and nutraceuticals.

### GOVERNMENT SUPPORT

This invention was made with government support under Grant Numbers AI130392 and GM136652, awarded by the National Institutes of Health. The Government has certain rights in the invention.

### RELATED APPLICATIONS

This application claims priority under 35 U.S.C. § 119(e) to U.S. Provisional Application, U.S.S.N. 63/503,914, filed May 23, 2023, the contents of which is incorporated herein by reference.

### BACKGROUND

Diet is instrumental in driving the composition and dynamics of the gut microbiome and in the development and prevention of human disease. Unlike the understanding of carbohydrate-microbe interactions, there is a dearth of knowledge as to phytochemical microbe interactions, whether these molecules are metabolized by enteric organisms, and how products of phytochemical catabolism affect microbiome composition or host physiology. Gut symbionts may leverage distinct genetic systems to transform dietary phytochemicals into bactericidal metabolites, and additional study of the molecular mechanisms of dietary phytochemical metabolism by dominant gut microorganisms and the effects of this metabolism on the maintenance of intestinal homeostasis is needed to inform the development of new therapeutics.

### SUMMARY

The present disclosure describes the identification of gut bacteria and their enzymes that metabolize dietary and medicinal plant-derived chemicals into bioactive chemicals that affect other gut bacteria (e.g., these metabolites kill pathogenic bacteria, such as Clostridioides difficile). As described herein, these microbes or their enzymes can be used to develop designer probiotics or personal diets for different conditions of health and disease. A number of gut bacteria species (including Bacteroides species, the dominant members in the western human gut microbiome) having previously uncharacterized proteins that metabolize chemicals that humans consume in diet and as medicine were identified. These bacteria use these enzymes to transform the chemical structures of these compounds (e.g., stilbene glycosides such polydatin and dihydrochalcone glycosides such as phloridzin) in a way that activates their bioactive effects. Gut or environmental bacterial enzymes that metabolize a fraction of these bioactive compounds have not been previously identified, nor have the effects of these bioactivities previously been demonstrated in vitro or in vivo. These enzymes are relatively simple and do not require other enzymes or chemical cofactors to function and can be expressed in other bacteria. The bacteria in which these enzymes are produced natively (i.e., Bacteroides species) are also the dominant bacteria in the human gut and can effectively metabolize these natural prodrugs (e.g., stilbene glycosides such polydatin and dihydrochalcone glycosides such as phloridzin) at the site where they are most needed (in the gut), allowing for targeted therapy of gut infections, including Clostridioides difficile infections.

Thus, in one aspect, the present disclosure provides pharmaceutical compositions comprising: (i) a microorganism capable of expressing an enzyme that converts a stilbene glycoside (e.g., polydatin) or a dihydrochalcone glycoside (e.g., phloridzin) to a stilbene (e.g., resveratrol) or a dihydrochalcone (e.g., phloretin), and (ii) a stilbene glycoside or a dihydrochalcone glycoside, or a pharmaceutically acceptable salt thereof.

In another aspect, the present disclosure provides methods for treating a Clostridioides difficile infection in a subject in need thereof, the method comprising administering to the subject a therapeutically effective amount of any of the pharmaceutical compositions provided herein.

In another aspect, the present disclosure provides methods for treating a Clostridioides difficile infection in a subject in need thereof, the method comprising administering to the subject a therapeutically effective amount of a pharmaceutical composition comprising a stilbene glycoside (e.g., polydatin) or a dihydrochalcone glycoside (e.g., phloridzin) with bactericidal activity against Clostridioides difficile when converted to a stilbene (e.g., resveratrol) or a dihydrochalcone (e.g., phloretin).

In another aspect, the present disclosure provides methods for treating a Clostridioides difficile infection in a subject in need thereof, the method comprising administering to the subject a therapeutically effective amount of a pharmaceutical composition comprising a stilbene (e.g., resveratrol) or a dihydrochalcone (e.g., phloretin) with bactericidal activity against Clostridioides difficile.

In another aspect, the present disclosure provides food products comprising (i) a microorganism capable of expressing an enzyme that converts a stilbene glycoside or a dihydrochalcone glycoside to a stilbene or a dihydrochalcone; (ii) a stilbene glycoside or a dihydrochalcone glycoside, or a pharmaceutically acceptable salt thereof; and (iii) a food.

In another aspect, the present disclosure provides nutraceuticals comprising (i) a microorganism capable of expressing an enzyme that converts a stilbene glycoside or a dihydrochalcone glycoside to a stilbene or a dihydrochalcone; (ii) a stilbene glycoside or a dihydrochalcone glycoside, or a pharmaceutically acceptable salt thereof; (iii) a food; and (iv) a dietary supplement.

### DEFINITIONS

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The term “administer,” “administering,” or “administration” refers to implanting, absorbing, ingesting, injecting, inhaling, or otherwise introducing a composition described herein in or on a subject. In some embodiments, compositions described herein are administered orally (e.g., ingested).

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference.

Pharmaceutically acceptable salts of the compounds of this disclosure (e.g., stilbene glycosides such polydatin and dihydrochalcone glycosides such as phloridzin) include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. In some embodiments, a subject has a Clostridioides difficile infection or is suspected of having a Clostridioides difficile infection.

A “therapeutically effective amount” of a composition described herein is an amount sufficient to provide a therapeutic benefit in the treatment of a condition (e.g., a Clostridioides difficile infection) or to delay or minimize one or more symptoms associated with the condition. A therapeutically effective amount of a composition means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for treating a Clostridioides difficile infection.

The terms “treatment,” “treat,” and “treating” refer to reversing, alleviating, delaying the onset of, or inhibiting the progress of a disease, disorder, or infection described herein, such as a Clostridioides difficile infection. In some embodiments, treatment may be administered after one or more signs or symptoms of the disease, disorder, or infection have developed or have been observed. In other embodiments, treatment may be administered in the absence of signs or symptoms of the disease, disorder, or infection. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

### BRIEF DESCRIPTION OF THE DRAWINGS

FIG. 1 shows the diversity of phytochemical small molecule prodrugs. Examples of natural polyphenol prodrugs from diet (drug-like aglycone with sugar masking group) are shown, including examples of stilbene glycosides and dihydrochalcone glycosides that can be converted to stilbenes and dihydrochalcones by enzymatic removal of a sugar moiety.

FIG. 2 shows that the symbiotic gut bacteria B. uniformis liberates phloretin and resveratrol from their natural prodrugs in vitro. Comparative LC-MS EICs (Extracted Ion Chromatogram) of methanol-water extracts of bacterial cultures supplemented with each polyphenolic glycoside after eight hours are provided. The trace labeled “A” corresponds to the EIC of each glycoside, and the trace labeled “B” corresponds to the EIC of each aglycone. The portion of the trace “B” peak overlapping the trace “A” peak corresponds to the aglycone detected from fragmentation of the glycoside at the ionization source and is a technical artifact.

FIGS. 3A-3C show that the microbial-liberated aglycones resveratrol and phloretin selectively kill the enteric pathogen C. difficile.Phloretin and resveratrol selectively kill hypervirulent C. difficile.Their natural prodrugs do not kill C. difficile.FIG. 3A provides a heatmap of the impact of polyphenol glycoside and aglycones on growth of a panel of gut bacterial pathogens. Growth is measured as the ratio of growth in media (AUC) supplemented with glucose and 150 μM glycoside or aglycone versus growth in media (AUC) supplemented with glucose and vehicle. Value>1 is representative of enhanced growth in compound and value<1 is representative of inhibition of growth in compound.

FIG. 3B provides a heatmap of growth of 20 Clostridioides difficile isolates in the absence of presence of the polyphenolic glycoside polydatin or the Bacteroides liberated aglycone resveratrol. Liberated aglycone resveratrol showed growth inhibition of Clostridioides difficile, while polyphenolic glycoside polydatin did not. Growth induction or inhibition is measured as the ratio of growth in media (AUC) supplemented with glucose and 150 μM polydatin or resveratrol versus growth in media (AUC) supplemented with glucose and vehicle. Value>1 is representative of enhanced growth in compound and value <1 is representative of inhibition of growth in compound. FIG. 3C shows the amount of C. difficile (colony forming units) at day one and day two in mice infected with C. difficile 630 treated with vehicle or 500 μM resveratrol or phloretin in their drinking water.

FIG. 4 shows that microbial-liberated resveratrol kills many diverse C. difficile isolates. Resveratrol showed growth inhibition of C. difficile, while polydatin did not. Bacterial isolates were grown in vitro in media plus compound. Killing was measured by bacterial culture optical density (proxy for amount of bacteria in a culture).

FIGS. 5A and 5B show microbiome-mediated polyphenol aglycone liberation. FIG. 5A provides growth curves showing that the microbial-liberated aglycones resveratrol and phloretin selectively kill the enteric pathogen C. difficile.Step 1: B. uniformis WT (metabolizer) was grown with either polydatin or vehicle. Step 2: Spent media was filter-sterilized. Step 3: C. difficile was grown in spent media from either vehicle or polydatin (top). Step 4: Step 3 was repeated with B. uniformis mutant lacking the ability to metabolize polydatin (bottom). Bacteroides bioactivation of polydatin to resveratrol and conditioned media from this culture inhibited the growth of Clostridioides difficile in vitro. FIG. 5B shows the percent conversion in vitro of polydatin to resveratrol by B. uniformis WT or B. uniformis mutant lacking the ability to metabolize polydatin (AgshD).

FIGS. 6A-6B show that administration of pure resveratrol or phloretin, the Bacteroides bioactivation products of polydatin and phloridzin, respectively, decreases C. difficile burden in an in vivo model of pathogen infection.

FIG. 7 shows that stilbenes (including resveratrol) represent a promising anti-C. difficile small molecule scaffold with potential therapeutic translation. Other natural and synthetic stilbene derivatives have enhanced bactericidal activity towards C. difficile.

FIG. 8 shows that stilbenes (including resveratrol) represent a promising anti-C. difficile small molecule scaffold with therapeutic applications. Several natural stilbene derivates had greater bactericidal activity compared to resveratrol. This activity is conserved against a broad panel of diverse C. difficile isolates.

### DETAILED DESCRIPTION

Delivery of stilbene glycosides (e.g., polydatin) or dihydrochalcone glycosides (e.g., phloridzin) plus Bacteroides as a synbiotic therapeutic is a novel therapeutic modality for targeted activation of medicinal small molecules in the colon where the Bacteroides are predominant, (1) lessening the need to dose at high levels to saturate system and (2) potentially preventing off-target effects. Engineering of any Bacteroides with enzymes specific to novel stilbene glycoside or dihydrochalcone glycoside derivatives would enable targeted metabolism of distinct substrates in the lower gut depending on probiotic consumed, allowing for combinatorial activation of anti-Clostridioides difficile small molecules with potentially different mechanisms of action/effects. Bacteroides as probiotic scaffolds are appealing as they are (1) oxygen-resistant and genetically tractable organisms, they (2) grow on diverse carbohydrates (enabling potential engraftment on specific diets) and they are (3) dominant members of the natural gut microbiome (unlike E. coli, for example), suggesting they might colonize the gut more robustly/resiliently over time, requiring less regular dosing over time. Thus, the present disclosure provides a novel suite of bacterial and enzymatic deliverables with direct translatability as synbiotic therapeutics to treat Clostridioides difficile infections.

### Pharmaceutical Compositions and Kits

In one aspect, the present disclosure provides pharmaceutical compositions comprising (i) a microorganism capable of expressing an enzyme that converts a stilbene glycoside or a dihydrochalcone glycoside to a stilbene or a dihydrochalcone (e.g., gshD from a species of Bacteroides), and (ii) a stilbene glycoside or a dihydrochalcone glycoside, or a pharmaceutically acceptable salt thereof. A microorganism is “capable of expressing” an enzyme if it contains genetic material (e.g., in the genome of the microorganism or on a heterologous expression vector, such as a plasmid) that encodes the enzyme. In some embodiments, the present disclosure contemplates the use of microorganisms that express an enzyme that converts a stilbene glycoside or a dihydrochalcone glycoside to a stilbene or a dihydrochalcone.

In some embodiments, the enzyme converts a dihydrochalcone glycoside to a dihydrochalcone. In some embodiments, the enzyme converts a stilbene glycoside to a stilbene. In some embodiments, the dihydrochalcone glycoside is phloridzin. In some embodiments, the stilbene glycoside is polydatin. In some embodiments, the dihydrochalcone glycoside is converted to phloretin. In some embodiments, the stilbene glycoside is converted to resveratrol. In some embodiments, a pharmaceutical composition further comprises a pharmaceutically acceptable carrier or buffer (e.g., a pharmaceutically acceptable excipient). In some embodiments, a pharmaceutical composition is formulated to dissolve in the digestive or GI tract of a subject. In some embodiments, a pharmaceutical composition is formulated to dissolve at a pH of about 6.8.

Various microorganisms may be included in the pharmaceutical compositions described herein. In some embodiments, the microorganism is a bacterium. In certain embodiments, the microorganism is not genetically engineered to express the enzyme that converts a stilbene glycoside or a dihydrochalcone glycoside to a stilbene or a dihydrochalcone. In some embodiments, the microorganism is a bacterium present in the human gut microbiome. In some embodiments, the microorganism is a species of Bacteroides (e.g., Bacteroides uniformis or Bacteroides ovatus).In certain embodiments, the microorganism is genetically engineered to heterologously express the enzyme that converts a stilbene glycoside or a dihydrochalcone glycoside to a stilbene or a dihydrochalcone. In some embodiments, the microorganism is lyophilized.

In some embodiments, a pharmaceutical composition is a pill, tablet, syrup, or solution. In some embodiments, a pharmaceutical composition is a pill or tablet. In certain embodiments, a pharmaceutical composition is a pill. In some embodiments, a pharmaceutical composition is a tablet. In some embodiments, a pharmaceutical composition is capable of local delivery to the digestive or GI tract of a subject. A pharmaceutical composition is “capable of local delivery” to the digestive or GI tract if it is formulated for oral administration (e.g., as a pill, tablet, syrup, or solution) or otherwise comprises a pharmaceutically acceptable excipient predisposing it for delivery to the digestive or GI tract. In some embodiments, a pharmaceutical composition comprises one or more pharmaceutically acceptable excipients that chemically and/or structurally predispose the pharmaceutical composition for delivery to the digestive or GI tract of a subject.

Pharmaceutically acceptable excipients that chemically and/or structurally predispose a pharmaceutical composition for delivery to the digestive or GI tract of a subject are known in the art and include, for example, those described in Hua, S. Advances in Oral Drug Delivery for Regional Targeting in the Gastrointestinal Tract-Influence of Physiological, Pathophysiological and Pharmaceutical Factors. Front. Pharmacol. 2020, 11, 1-22, which is incorporated herein by reference. In some embodiments, pharmaceutically acceptable excipients that chemically and/or structurally predispose a pharmaceutical composition for delivery to the digestive or GI tract of a subject include cellulose acetate phthalate, cellulose acetate trimellitate, hydroxypropyl methylcellulose phthalate 55, hydroxypropyl methylcellulose phthalate 50, and/or polyvinyl acetate phthalate.

In some embodiments, the microorganism converts the stilbene glycoside or the dihydrochalcone glycoside to the stilbene or the dihydrochalcone. In some embodiments, the microorganism converts phloridzin to phloretin. In some embodiments, the microorganism converts polydatin to the resveratrol. In certain embodiments, the microorganism does not convert the stilbene glycoside or the dihydrochalcone glycoside to the stilbene or the dihydrochalcone until the pharmaceutical composition has been delivered to the digestive or GI tract of a subject.

In certain embodiments, a pharmaceutical composition described herein is provided in an effective amount to a subject. In certain embodiments, the effective amount is a therapeutically effective amount. In certain embodiments, the effective amount is a prophylactically effective amount. In certain embodiments, the effective amount is an amount effective for treating a C. difficile infection in a subject in need thereof. In certain embodiments, the effective amount is an amount effective for preventing the development of a C. difficile infection in a subject in need thereof. In certain embodiments, the effective amount is an amount effective for reducing the risk of developing a C. difficile infection in a subject in need thereof.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

Pharmaceutical compositions described herein can be prepared by any method known in the art of pharmaceutics. In general, such preparatory methods include bringing the components of the composition described herein (i.e., the “active ingredients”) into association with a carrier or excipient, and/or one or more other accessory ingredients, and then, if necessary and/or desirable, shaping, and/or packaging the product into a desired single-or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

The compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical, mucosal, nasal, bucal, sublingual; by intratracheal instillation, bronchial instillation, and/or inhalation; and/or as an oral spray, nasal spray, and/or aerosol.

A composition, as described herein, can be administered in combination with one or more additional pharmaceutical agents (e.g., therapeutically and/or prophylactically active agents). The compositions can be administered in combination with additional pharmaceutical agents that improve their activity (e.g., activity (e.g., potency and/or efficacy) in treating a disease or infection in a subject in need thereof, in preventing a disease or infection in a subject in need thereof, and/or in reducing the risk of developing a disease or infection in a subject in need thereof. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the composition described herein in a single dose or composition or administered separately in different doses or compositions. The particular combination to employ in a regimen will take into account compatibility of the composition described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

Also encompassed by the disclosure are kits (e.g., pharmaceutical packs). The kits provided may comprise a pharmaceutical composition described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition described herein. In some embodiments, the pharmaceutical composition described herein provided in the first container and the second container are combined to form one unit dosage form.

Thus, in one aspect, provided are kits including a first container comprising a pharmaceutical composition described herein. In certain embodiments, the kits are useful for treating a disease, disorder, or infection (e.g., a C. difficile infection) in a subject in need thereof. In certain embodiments, the kits are useful for preventing a disease, disorder, or infection (e.g., a C. difficile infection) in a subject in need thereof. In certain embodiments, the kits are useful for reducing the risk of developing a disease, disorder, or infection (e.g., a C. difficile infection) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease, disorder, or infection (e.g., a C. difficile infection) in a subject in need thereof. In certain embodiments, the kits and instructions provide for preventing a disease, disorder, or infection (e.g., a C. difficile infection) in a subject in need thereof. In certain embodiments, the kits and instructions provide for reducing the risk of developing a disease, disorder, or infection (e.g., a C. difficile infection) in a subject in need thereof. A kit described herein may include one or more additional pharmaceutical agents described herein as a separate composition.

### Methods for Treating a Clostridioides Difficile Infection

In another aspect, the present disclosure provides methods for treating a Clostridioides difficile infection in a subject in need thereof, the method comprising administering to the subject a therapeutically effective amount of any of the pharmaceutical compositions provided herein.

In another aspect, the present disclosure provides methods for treating a Clostridioides difficile infection in a subject in need thereof, the method comprising administering to the subject a therapeutically effective amount of a pharmaceutical composition comprising a stilbene glycoside (e.g., polydatin) or a dihydrochalcone glycoside (e.g., phloridzin) with bactericidal activity against Clostridioides difficile when converted to a stilbene (e.g., resveratrol) or a dihydrochalcone (e.g., phloretin).

In another aspect, the present disclosure provides methods for treating a Clostridioides difficile infection in a subject in need thereof, the method comprising administering to the subject a therapeutically effective amount of a pharmaceutical composition comprising a stilbene (e.g., resveratrol) or a dihydrochalcone (e.g., phloretin) with bactericidal activity against Clostridioides difficile.

In some embodiments, the subject is a human. In some embodiments, the pharmaceutical composition is administered to the subject orally. In some embodiments, treatment of the Clostridioides difficile infection is effected through conversion of the stilbene glycoside or the dihydrochalcone glycoside to a stilbene or a dihydrochalcone by a microorganism in the digestive or GI tract of the subject.

### Food Products and Nutraceuticals

In other aspects, the present disclosure provides food products and nutraceuticals.

In one aspect, the present disclosure provides food products comprising (i) a microorganism capable of expressing an enzyme that converts a stilbene glycoside or a dihydrochalcone glycoside to a stilbene or a dihydrochalcone; (ii) a stilbene glycoside or a dihydrochalcone glycoside, or a pharmaceutically acceptable salt thereof; and (iii) a food.

In another aspect, the present disclosure provides nutraceuticals comprising (i) a microorganism capable of expressing an enzyme that converts a stilbene glycoside or a dihydrochalcone glycoside to a stilbene or a dihydrochalcone; (ii) a stilbene glycoside or a dihydrochalcone glycoside, or a pharmaceutically acceptable salt thereof; (iii) a food; and (iv) a dietary supplement.

### EXAMPLES

In order that the present disclosure may be more fully understood, examples are set forth. The examples described in this application are offered to illustrate the methods, products, and compositions provided herein and are not to be construed in any way as limiting their scope.

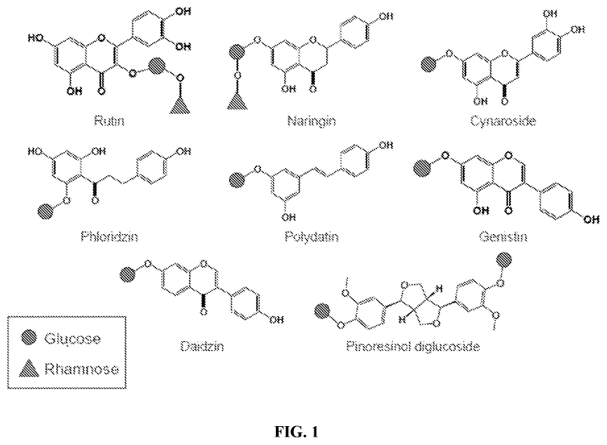

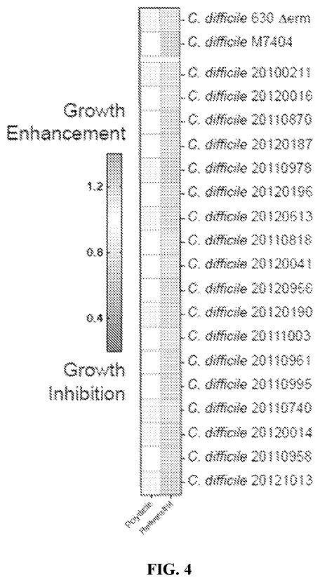

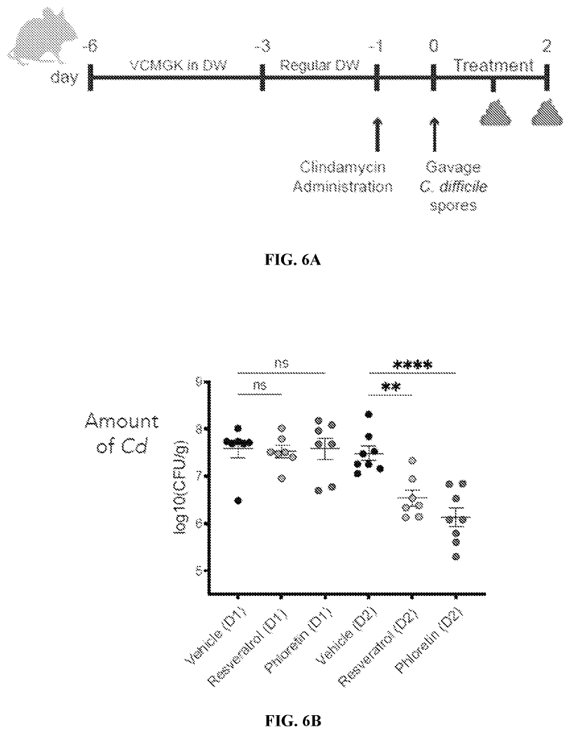

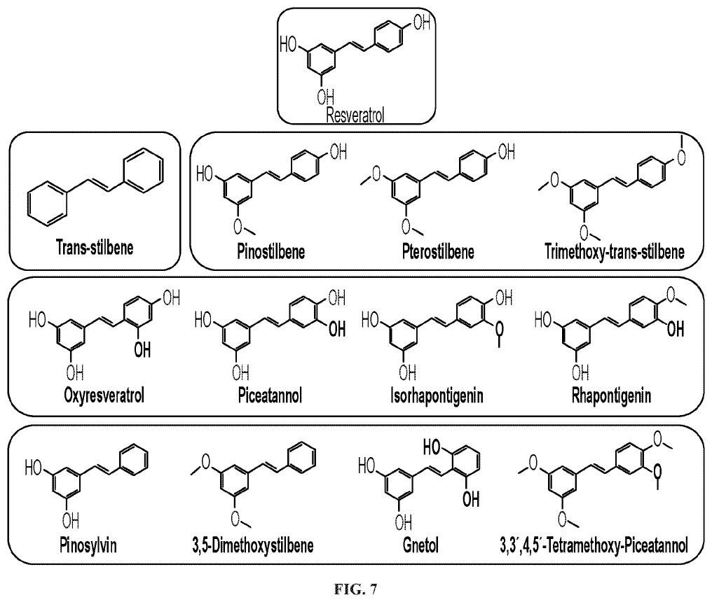

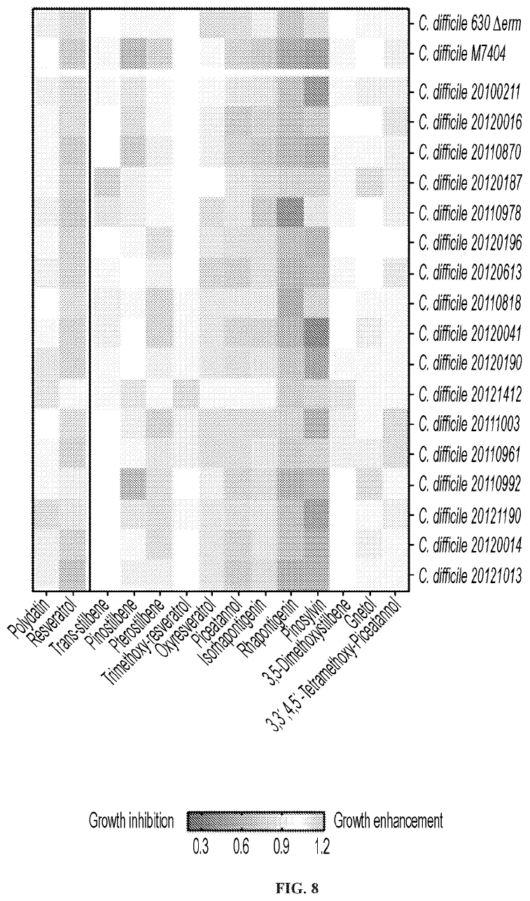

The present disclosure identifies and characterizes a novel mechanism by which the gut microbiome transforms small molecule components of human diet to drive pro-homeostatic programs in the intestine. Experiments demonstrating and enabling the practice of the subject matter of the present disclosure are described in FIGS. 1-8.

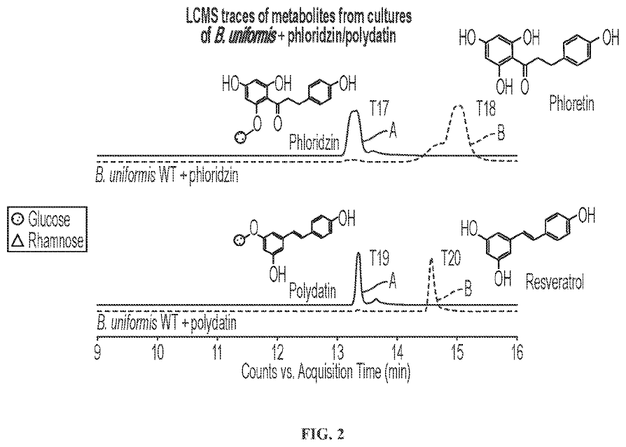

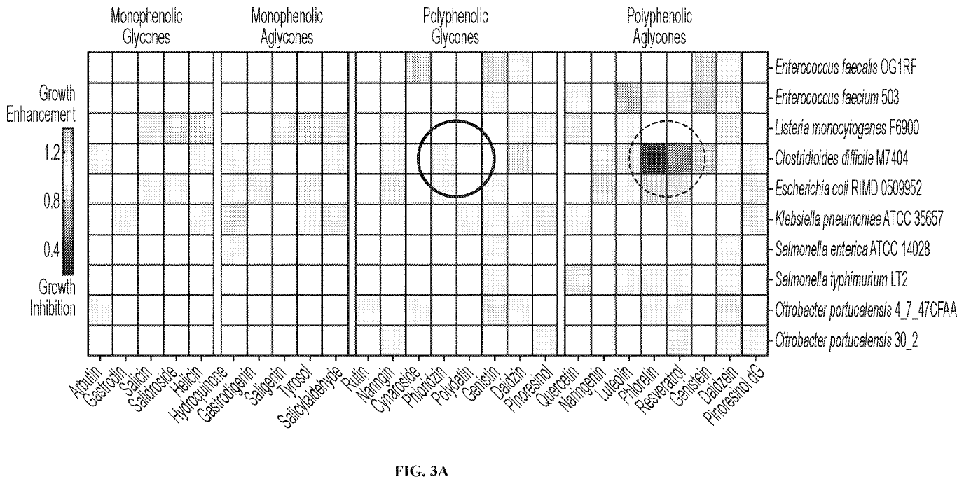

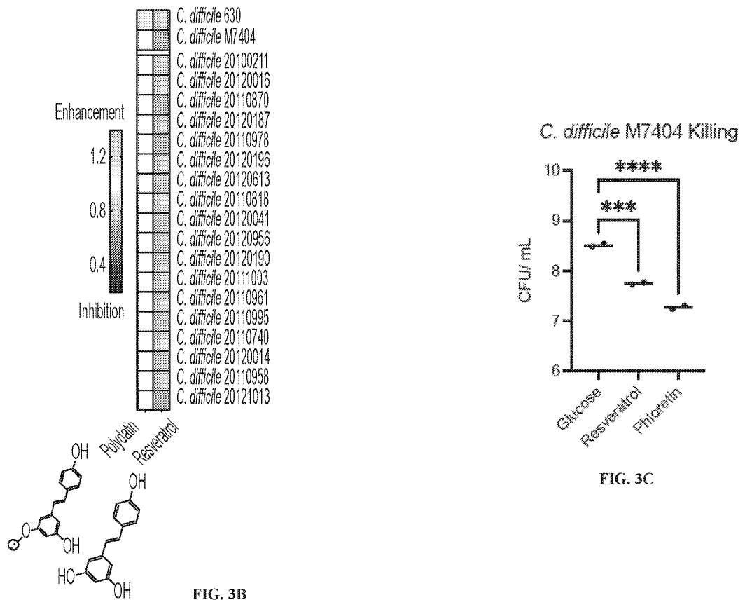

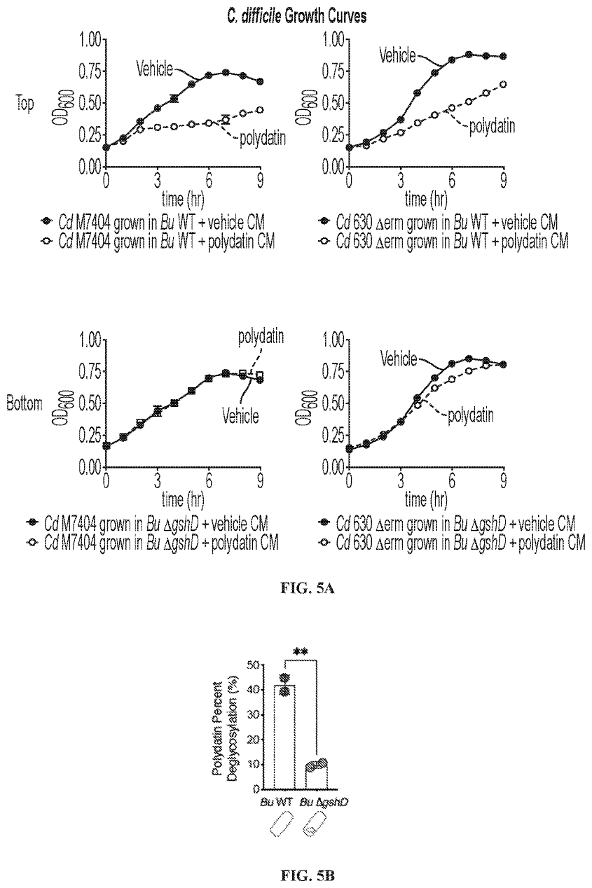

B. uniformis cultivated with polydatin liberated resveratrol (FIG. 2) and generated potent anti-C.difficile activity (FIG. 5A). Both functions were ablated in B. uniformis AgshD (FIGS. 5A and 5B), demonstrating bioactivation of polydatin and liberation of resveratrol by B. uniformis dependent on the glycoside-specific GH 3 Bu gshD. C. difficile infection is a major cause of mortality and morbidity, most associated with defects in colonization resistance by the gut microbiome in the setting of use of antibiotics. Across 20 unique C. difficile strains representing toxin production and hypervirulence, it was found that the B. uniformis-liberated polyphenolic aglycone resveratrol, but not its parent glycoside polydatin, as universally anti-microbial against C. difficile (FIG. 3B). In a mouse model of C. difficile infection, the B. uniformis generated aglycones resveratrol and phloretin demonstrated potent anti-microbial effects on C. difficile in vivo (FIG. 3C). Together, the present disclosure reveals both a novel function of polyphenolic aglycones against the human gut pathogen C. difficile and the role of bioactivation of dietary plant glycosides by members of the gut microbiome in mediating a novel mechanism of colonization resistance to human gut pathogens mediated by microbiome bioactivation of specific phytochemicals.

### Materials and Methods

Bacterial culture: All human gut bacterial strains were purchased from the American Type Culture Collection (ATCC), Deutsche Sammlung von Mikroorganismen und Zellkulturen GmbH (DSMZ), the Biodefense and Emerging Infections Research Resources Repository (BEI resources), or from collaborators specified above, except for those strains generated in this work. Frozen stock cultures of bacteria were stored at −80 °C in cryogenic vials. Individual strains were derived from the same glycerol stocks throughout this Example.

Gut bacterial glycoside utilization: All Bacteroidetes isolates were grown on brain-heart-infusion supplemented with hemin (50 mg/L) and vitamin K1 (0.25 mg/L) (BHIS) agar plates or in YBHIS (brain-heart-infusion powder (37 g/L), yeast (5 g/L), D-(+)-cellobiose (1 g/L), D-(+)-maltose monohydrate (1 g/L), cysteine (0.5 g/L), hemin (50 mg/L), and vitamin K1 (0.25 mg/L). To determine the ability of diverse gut bacteria to utilize glucose (Sigma) or phytochemical glycosides (salicin (Sigma), arbutin (Sigma), salidroside (ChemImpex), gastrodin (TCI), helicin (TCI), esculin (Sigma), amygdalin (Sigma)) as a sole carbon source, each isolate was recovered on a BHIS or BBA agar plate and incubated anaerobically at 37 °C for 48 h. A starter culture (1 mL) of YBHIS was inoculated with each isolate and grown for 15 h. This starter culture was then used to inoculate a YBHIS sub-culture (1 mL) at a final dilution of 1:10. This sub-culture was grown for 3 h to an OD600~0.5 before being used to inoculate experimental cultures at a final dilution of 1:50. The experimental media consisted of a modified YCFA media (mYCFA; casitone (2.5 g/L), yeast extract (0.625 g/L), cysteine (0.5 g/L), magnesium sulfate heptahydrate (f.c. 365 μM), calcium chloride dihydrate (f.c. 610 μM), sodium bicarbonate (4 g/L), dipotassium phosphate (0.45 g/L), monopotassium phosphate (0.45 g/L), sodium chloride (0.9 g/L), sodium acetate (2.71 g/L), Hemin (50 mg/L), Vitamin K1 (0.25 mg/L), ferrous sulfate (4 μg/mL), ATCC vitamin mix (1% v/v)) supplemented with glucose (15 mM, Sigma) or glycoside (15 mM). Bacterial growth was monitored by measuring the optical density at 600 nm (OD₆₀₀) of each culture in a 384 well plate with an Epoch2 spectrophotometer (BioTek Instruments) and MicroPlate stacker (Agilent BioTek) for 24 hours.

Gut Bacteroides glycoside utilization: All Bacteroidetes isolates were grown on brain-heart-infusion supplemented with hemin (50 mg/L) and vitamin K1 (0.25 mg/L) (BHIS) agar plates or in basal media (BSG; proteose peptone (20 g/L), yeast (5 g/L), NaCl (5 g/L), glucose (5 g/L), potassium phosphate dibasic (5 g/L), cysteine (0.5 g/L), hemin (50 mg/L), and vitamin K1 (0.25 mg/L). All isolates were recovered on and grown in pre-reduced media in a Coy anaerobic chamber (Coy Labs). To determine the ability of Bacteroidetes strains to utilize glucose (Sigma), phytochemical glycosides (salicin (Sigma), arbutin (Sigma), salidroside (ChemImpex), gastrodin (TCI), helicin (TCI), esculin (Sigma), amygdalin (Sigma)), or disaccharides (D-(+)-cellobiose (Sigma), lactose (Sigma), D-(+)-maltose monohydrate (Sigma), melibiose (Sigma), sucrose (Sigma), trehalose (Sigma), palatinose (TCI), gentiobiose (TCI)) as a sole carbon source, each isolate was recovered on a BHIS agar plate and incubated anaerobically at 37 °C for 48 h. A starter culture (1 mL) of BSG was inoculated with each isolate and grown for 15 h. This starter culture was then used to inoculate a BSG sub-culture (1 mL) at a final dilution of 1:10. This sub-culture was grown for 3 h to an OD600~0.5 before being used to inoculate experimental cultures at a final dilution of 1:50. The experimental media consisted of Bacteroides minimal media (BMM; ammonium sulfate (1 g/L), sodium carbonate (1 g/L), potassium phosphate monobasic (0.9 g/L), sodium chloride (0.9 g/L), calcium chloride dihydrate (26.5 mg/L), magnesium chloride hexahydrate (2 mg/L), manganese(II) chloride tetrahydrate (1 mg/L), cobalt(II) chloride hexahydrate (1 mg/L), hemin (50 mg/L), vitamin K1 (0.25 mg/L), ferrous sulfate heptahydrate (4 mg/L), vitamin B12 (5 mg/L)) supplemented with glucose (15 mM, Sigma), glycoside (15 mM) or disaccharide (15 mM). Bacterial growth was monitored by measuring the optical density at 600 nm (OD₆₀₀) of each culture in a 384 well plate with an Epoch 2 spectrophotometer (BioTek Instruments) and MicroPlate stacker (Agilent BioTek) for 24 hours.

Generation of Bacteroides gene deletion mutants: Clean deletion mutants were created by allelic replacement whereby Bacteroides uniformis ATCC 8492 gshD (BACUNI_00919)was deleted. A modified pKNOCK-bla-ermGb suicide vector, pKNOCK-bla-ermGb_pheS*, that was previously engineered with a counter-selection system mediated by a variant phenylalanyl-tRNA synthetase (PheSA303G), lethal in the presence of 4-chloro-phenylalanine was leveraged. To generate deletion constructs, 1 kb regions flanking the genomic region-of-interest were amplified by PCR (Platinum SuperFi PCR Master Mix), purified, and cloned into the pKNOCK-bla-ermGb_pheS* plasmid in a three-piece ligation reaction. To mobilize resulting plasmids into Bacteroides, a triparental mating strategy was implemented. Overnight cultures of Bacteroides grown in BSG were diluted 50-fold into fresh pre-reduced BSG and grown to early exponential phase (OD₆₀₀ ~0.1). The recipient strain was centrifuged at 4,000 rpm for 5 minutes and resuspended in 200 μL of 1× PBS. One-day-old E. coliDH5α lambda pir donor strain and E. coliDH5α helper strain with pRK 231 were scrapped from LB agar plates into 200 μL of 1× PBS. Donor, helper, and recipient strains were combined at a 1:1:1 donor: helper: recipient culture volume ratio at a final volume of 90 μL and spotted on non-selective BHIS agar plate for 18 hours at 37 °C under aerobic conditions to allow for conjugation. Mating lawns were scrapped onto BHIS agar plates containing gentamicin (200 μg/mL) and erythromycin (5 μg/mL) to select for transconjugants (merodiploids). Single colonies were isolated by re-streaking colonies onto BHIS agar plates containing erythromycin (5 μg/mL). A single colony was re-streaked onto BMM agar plates supplemented with 4-Chloro-DL-phenylalanine (2 mg/mL, Acros Organics) to select for the loss of the pKNOCK-bla-tetQ-pheS* vector. Individual clones were replica-plated onto BHIS agar and BHIS agar supplemented with erythromycin (5 μg/mL) and those clones that did not grow on erythromycin were confirmed for genetic manipulation by PCR and sequencing.

In vitro aglycone liberation: All B. uniformis strains were grown on brain-heart-infusion supplemented with hemin (50 mg/L) and vitamin K1 (0.25 mg/L) (BHIS) agar plates or in basal media (BSG; proteose peptone (20 g/L), yeast (5 g/L), NaCl (5 g/L), glucose (5 g/L), potassium phosphate dibasic (5 g/L), cysteine (0.5 g/L), hemin (50 mg/L), and vitamin K1 (0.25 mg/L). All isolates were recovered on and grown in pre-reduced media in a Coy anaerobic chamber (Coy Labs).

To assess the capacity of Bacteroides to liberate aglycones from their parent aryl, coumarin, and cyanogenic glycosides, wildtype B. uniformis ATCC 8492 was recovered on a BHIS agar plate and incubated anaerobically at 37 °C for 48 h. A starter culture (1 mL) of BSG was inoculated with each isolate and grown for 15 h. This starter culture was then used to inoculate a BSG sub-culture (1 mL) at a final dilution of 1:10. This sub-culture was grown for 3 h to an OD600~0.5 before being used to inoculate experimental cultures at a final dilution of 1:50. The experimental media consisted of Bacteroides minimal media (BMM; ammonium sulfate (1 g/L), sodium carbonate (1 g/L), potassium phosphate monobasic (0.9 g/L), sodium chloride (0.9 g/L), calcium chloride dihydrate (26.5 mg/L), magnesium chloride hexahydrate (2 mg/L), manganese(II) chloride tetrahydrate (1 mg/L), cobalt(II) chloride hexahydrate (1 mg/L), hemin (50 mg/L), vitamin K1 (0.25 mg/L), ferrous sulfate heptahydrate (4 mg/L), vitamin B12 (5 mg/L)) supplemented with glucose (0.1%, Sigma) and any one G1G (500 μM, Sigma) or glucose (0.1%, Sigma) and DMSO (0.5%, Sigma). At late log, each experimental culture was isolated, diluted (1:50) into 80% methanol-water spiked with tyrosol-D4 (40 nM, TRC Canada), vortexed (2 min), centrifuged (5000 rpm, 5 min), and the supernatant was recovered for down-stream LC-MS analyses.

To assess the capacity of Bacteroides to liberate aglycones from their parent polyphenolic glycosides, B. uniformis ATCC 8492 WT and AgshD were recovered on a BHIS agar plate and incubated anaerobically at 37 °C for 48 h. A starter culture (1 mL) of BSG was inoculated with each isolate and grown for 15 h. This starter culture was then used to inoculate a BSG sub-culture (1 mL) at a final dilution of 1:10. This sub-culture was grown for 3 h to an OD600~0.5 before being used to inoculate experimental cultures at a final dilution of 1:50. The experimental media consisted of Bacteroides minimal media (BMM; ammonium sulfate (1 g/L), sodium carbonate (1 g/L), potassium phosphate monobasic (0.9 g/L), sodium chloride (0.9 g/L), calcium chloride dihydrate (26.5 mg/L), magnesium chloride hexahydrate (2 mg/L), manganese(II) chloride tetrahydrate (1 mg/L), cobalt(II) chloride hexahydrate (1 mg/L), hemin (50 mg/L), vitamin K1 (0.25 mg/L), ferrous sulfate heptahydrate (4 mg/L), vitamin B12 (5 mg/L)) supplemented with glucose (0.1%, Sigma) and any one G3G (500 μM, Sigma) or glucose (0.1%, Sigma) and DMSO (0.5%, Sigma). At late log, each experimental culture was isolated, diluted (1:50) into 80% methanol-water spiked with C¹³-Resveratrol (250 nM, Sigma), vortexed (2 min), centrifuged (5000 rpm, 5 min), and the supernatant was recovered for down-stream LC-MS analyses.

Glycoside and aglycone detection by LC/MS: For aryl glycoside and aglycone-containing samples, samples were dried under nitrogen flow and resuspended in 50 μL of methanol 50% in water. A standard curve was prepared using the same extraction solution and volumes as for the samples. The curve was prepared as a 10 points ⅕ dilution series with 1 mM as the highest concentration. Samples were quantified on a QEplus mass spectrometer coupled to an Ultimate 3000 LC (Thermo fisher). Five microliters were injected on a Luna Omega Polar C18 column (2 mm×150 mm, Phenomenex) maintained at 40 °C The mobile phases were A: water and B: Methanol. The gradient was as follows: 1% B for 4 min, then to 100% B in 6 min. the mobile phases were then maintained at 100% B for 15 min, followed by 5 min re-equilibration at 1% B. The flow rate was 0.15 mL min⁻¹. Ionization for the mass spectrometer was achieved with atmospheric pressure chemical ionization (APCI) with a probe at 350 °C, in switching polarity mode. Quantification was performed using Tracefinder (Thermo fisher), using the ratio of the area under the peak for each compound and of the internal standard, using the accurate mass of the [M−H]⁻ ions mainly, except for hydroquinone and benzoquinone where the [M+e]⁻ and the [M+H]⁺ were used respectively.

For polyphenolic glycoside and aglycone-containing samples, samples were dried under nitrogen flow and resuspended in 50 μl of methanol 50% in water. A standard curve was prepared using the same extraction solution as for the samples. The curve was prepared as a 10 points ⅕ dilution series with 100 μM as the highest concentration. Samples were quantified on a QEplus mass spectrometer coupled to an Ultimate 3000 LC (Thermo fisher). Five microliters were injected on a C18Evo column (2mm×150 mm, Phenomenex) maintained at 30 °C. The mobile phases were A: water, 5 mM ammonium formate, with pH adjusted to 7 and B: Acetonitrile, 0.1% ammonium hydroxide. The gradient was as follow: 0% B for 5 min, then to 40% B in 9 min, then to 50% B in 6 min, and finally to 100% B in 1 min. The mobile phases were then maintained at 100% B for 7 min, followed by 5 min re-equilibration at 0% B. The flow rate was 0.15 mL min⁻¹. Ionization was achieved by heated electrospray ionization (HESI) in negative mode. Quantification was performed using Tracefinder (Thermo fisher), using the ratio of the area under the peak for each compound and of the internal standard, using the accurate mass of the [M−H]⁻ ions.

Phytochemical microbial killing assays: All gut pathogen isolates were grown on brain-heart-infusion supplemented with hemin (50 mg/L) and vitamin K1 (0.25 mg/L) (BHIS) agar plates or in basal media (BSG; proteose peptone (20 g/L), yeast (5 g/L), NaCl (5 g/L), glucose (5 g/L), potassium phosphate dibasic (5 g/L), cysteine (0.5 g/L), hemin (50 mg/L), and vitamin K1 (0.25 mg/L). Isolates were recovered on and grown in pre-reduced media in a Coy anaerobic chamber (Coy Labs). To determine the fitness effects of phloridzin hydrate (TCI), phloretin (TCI), polydatin (TCI), and resveratrol (TCI) on enteric pathogen fitness, each pathogen isolate was recovered on a BHIS agar plate and incubated anaerobically at 37 °C for 48 h. A starter culture (1 mL) of BSG was inoculated with C. difficile and grown for 15 h. This starter culture was then used to inoculate a BSG sub-culture (1 mL) at a final dilution of 1:10. This sub-culture was grown for 3 h to an OD600~0.5 before being used to inoculate experimental cultures at a final dilution of 1:50. The experimental media consisted of a modified YCFA media (mYCFA; casitone (2.5 g/L), yeast extract (0.625 g/L), cysteine (0.5 g/L), magnesium sulfate heptahydrate (f.c. 365 μM), calcium chloride dihydrate (f.c. 610 μM), sodium bicarbonate (4 g/L), dipotassium phosphate (0.45 g/L), monopotassium phosphate (0.45 g/L), sodium chloride (0.9 g/L), sodium acetate (2.71 g/L), Hemin (50 mg/L), Vitamin K1 (0.25 mg/L), ferrous sulfate (4 μg/mL), ATCC vitamin mix (1% v/v)) supplemented with glucose (15 mM, Sigma) plus each compound (150 μM) or glucose (15 mM, Sigma) plus DMSO (0.15%, Sigma). Bacterial growth was monitored by measuring the optical density at 600 nm (OD₆₀₀) of each culture in a 384 well plate with an Epoch2 spectrophotometer (BioTek Instruments) and MicroPlate stacker (Agilent BioTek) for 24 hours.

In vitro polydatin bioactivation and C. difficile antagonism: B. uniformis strains were grown on brain-heart-infusion supplemented with hemin (50 mg/L) and vitamin K1 (0.25 mg/L) (BHIS) agar plates or in basal media (BSG; proteose peptone (20 g/L), yeast (5 g/L), NaCl (5 g/L), glucose (5 g/L), potassium phosphate dibasic (5 g/L), cysteine (0.5 g/L), hemin (50 mg/L), and vitamin K1 (0.25 mg/L). Isolates were recovered on and grown in pre-reduced media in a Coy anaerobic chamber (Coy Labs). To assess the effects of resveratrol liberation from polydatin on the fitness of C. difficile M7404, wildtype B. uniformis ATCC 8492 and B. uniformis ATCC 8492 AgshD were recovered on BHIS agar plates and incubated anaerobically at 37 °C for 48 h. A starter culture (1 mL) of BSG was inoculated with each isolate and grown for 15 h. This starter culture was then used to inoculate a BSG sub-culture (1 mL) at a final dilution of 1:10. This sub-culture was grown for 3 h to an OD₆₀₀ ~0.5 before being used to inoculate experimental cultures at a final dilution of 1:50. The experimental media consisted of Bacteroides minimal media (BMM; ammonium sulfate (1 g/L), sodium carbonate (1 g/L), potassium phosphate monobasic (0.9 g/L), sodium chloride (0.9 g/L), calcium chloride dihydrate (26.5 mg/L), magnesium chloride hexahydrate (2 mg/L), manganese(II) chloride tetrahydrate (1 mg/L), cobalt(II) chloride hexahydrate (1 mg/L), hemin (50 mg/L), vitamin K1 (0.25 mg/L), ferrous sulfate heptahydrate (4 mg/L), vitamin B12 (5 mg/L)) supplemented with glucose (0.1%, Sigma) and polydatin (500 μM, Sigma) or glucose (0.1%, Sigma) and DMSO (0.5%, Sigma). After 8 hours, conditioned media was prepared from experimental cultures by centrifugation (5000 rpm, 5 min), and the supernatant was recovered and filter-sterilized (0.22 μm).

To assess the capacity of B. uniformis metabolism of polydatin to antagonize the fitness of C. difficile, C. difficile M7404 was recovered on a pre-reduced BHIS agar plate and incubated anaerobically at 37 °C for 48 h. A starter culture (1 mL) of BSG was inoculated with C. difficile and grown for 15 h. This starter culture was then used to inoculate a BSG sub-culture (1 mL) at a final dilution of 1:10. This sub-culture was grown for 3 h to an OD600~0.5 before being used to inoculate experimental cultures at a final dilution of 1:50.

The experimental media consisted of a modified YCFA media (mYCFA; casitone (2.5 g/L), yeast extract (0.625 g/L), cysteine (0.5 g/L), magnesium sulfate heptahydrate (f.c. 365 μM), calcium chloride dihydrate (f.c. 610 μM), sodium bicarbonate (4 g/L), dipotassium phosphate (0.45 g/L), monopotassium phosphate (0.45 g/L), sodium chloride (0.9 g/L), sodium acetate (2.71 g/L), Hemin (50 mg/L), Vitamin K1 (0.25 mg/L), ferrous sulfate (4 μg/mL), and ATCC vitamin mix (1% v/v)) supplemented with glucose (15 mM, Sigma) plus ½-diluted B. uniformis WT or AgshD conditioned media from growth with or without polydatin. C. difficile growth was monitored by measuring the optical density at 600 nm (OD₆₀₀) of each culture in a 384 well plate with an Epoch2 spectrophotometer (BioTek Instruments) for 12 hours.

C. difficile infection (CDI) model: An antibiotic-treatment-induced CDI mouse model developed previously was used for the infection model. Briefly, six-to eight-week-old male and female SPF C57BL/6J WT mice were treated for three days with a mixture of antibiotics including vancomycin (0.4 mg/mL, Sigma) colistin (850 U/mL, Sigma), metronidazole (0.215 mg/mL, Sigma), gentamicin (0.035 mg/mL, RPI), and kanamycin (0.045 mg/mL, Sigma) in their drinking water. Mice were then treated with regular water for two days and then injected intraperitoneal (IP) with clindamycin (10 mg/kg, Mylan Pharmaceuticals) in normal saline. After one day, mice were gavaged with 104 spores of C. difficile M7404 diluted in PBS. Stool was collected from mice at one day and two days post infection, and immediately taken for assessment of C. difficile infection load.

C. difficile colony-forming units (CFUs): Mouse feces were collected from mice on day one and day two of C. difficile infection. Feces were weighed and resuspended to 50 mg/mL in pre-reduced 1× PBS. CFUs were enumerated by plating multiple dilutions on pre-reduced commercial ChromID C. difficile -selective plates (Biomerieux) after incubation for 24 hours at 37 °C in an anaerobic chamber (Coy Labs).

### Equivalents And Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included.

Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A pharmaceutical composition comprising:
(i) a microorganism capable of expressing an enzyme that converts a stilbene glycoside or a dihydrochalcone glycoside to a stilbene or a dihydrochalcone, and
(ii) a stilbene glycoside or a dihydrochalcone glycoside, or a pharmaceutically acceptable salt thereof.

2. The pharmaceutical composition of claim 1, wherein the enzyme converts a stilbene glycoside to a stilbene.

3. The pharmaceutical composition of claim 1, wherein the enzyme converts the dihydrochalcone glycoside to the dihydrochalcone.

4. The pharmaceutical composition of claim 1, wherein the dihydrochalcone glycoside is phloridzin.

5. The pharmaceutical composition of claim 1, wherein the stilbene glycoside is polydatin.

6. The pharmaceutical composition of claim 1, wherein the dihydrochalcone glycoside is converted to phloretin.

7. The pharmaceutical composition of claim 1, wherein the stilbene glycoside is converted to resveratrol.

8. The pharmaceutical composition of any one of claims 1-7 further comprising a pharmaceutically acceptable carrier or buffer.

9. The pharmaceutical composition of any one of claims 1-8, wherein the pharmaceutical composition is formulated to dissolve in the digestive or GI tract of a subject.

10. The pharmaceutical composition of any one of claims 1-9, wherein the pharmaceutical composition is formulated to dissolve at a pH of about 6.8.

11. The pharmaceutical composition of any one of claims 1-10, wherein the microorganism is a bacterium.

12. The pharmaceutical composition of any one of claims 1-11, wherein the microorganism is not genetically engineered to express the enzyme that converts a stilbene glycoside or a dihydrochalcone glycoside to a stilbene or a dihydrochalcone.

13. The pharmaceutical composition of any one of claims 1-12, wherein the microorganism is a bacterium from the human gut microbiome.

14. The pharmaceutical composition of any one of claims 1-13, wherein the microorganism is a species of Bacteroides.

15. The pharmaceutical composition of any one of claims 1-14, wherein the microorganism is Bacteroides uniformis.

16. The pharmaceutical composition of any one of claims 1-14, wherein the microorganism is Bacteroides ovatus.

17. The pharmaceutical composition of any one of claims 1-11 or 13-16, wherein the microorganism is genetically engineered to heterologously express the enzyme that converts a stilbene glycoside or a dihydrochalcone glycoside to a stilbene or a dihydrochalcone.

18. The pharmaceutical composition of any one of claims 1-17, wherein the microorganism is lyophilized.

19. The pharmaceutical composition of any one of claims 1-18, wherein the enzyme that converts a stilbene glycoside or a dihydrochalcone glycoside to a stilbene or a dihydrochalcone is gshD from Bacteroides uniformis.

20. The pharmaceutical composition of any one of claims 1-19, wherein the pharmaceutical composition is a pill, tablet, syrup, or solution.

21. The pharmaceutical composition of any one of claims 1-20, wherein the pharmaceutical composition is capable of local delivery to the digestive or GI tract of a subject.

22. The pharmaceutical composition of any one of claims 1-21, wherein the pharmaceutical composition comprises one or more pharmaceutically acceptable excipients that chemically and/or structurally predispose the pharmaceutical composition for delivery to the digestive or GI tract of a subject.

23. The pharmaceutical composition of any one of claims 1-22, wherein the microorganism converts the stilbene glycoside or the dihydrochalcone glycoside to the stilbene or the dihydrochalcone.

24. The pharmaceutical composition of any one of claims 1-23, wherein the microorganism converts phloridzin to phloretin.

25. The pharmaceutical composition of any one of claims 1-23, wherein the microorganism converts polydatin to resveratrol.

26. The pharmaceutical composition of claim 23, wherein the microorganism does not convert the stilbene glycoside or the dihydrochalcone glycoside to the stilbene or the dihydrochalcone until the pharmaceutical composition has been delivered to the digestive or GI tract of a subject.

27. A method for treating a Clostridioides difficile infection in a subject in need thereof, the method comprising administering to the subject a therapeutically effective amount of the pharmaceutical composition of any one of claims 1-26.

28. A method for treating a Clostridioides difficile infection in a subject in need thereof, the method comprising administering to the subject a therapeutically effective amount of a pharmaceutical composition comprising a stilbene glycoside or a dihydrochalcone glycoside with bactericidal activity against Clostridioides difficile when converted to a stilbene or a dihydrochalcone.

29. A method for treating a Clostridioides difficile infection in a subject in need thereof, the method comprising administering to the subject a therapeutically effective amount of a pharmaceutical composition comprising a stilbene or a dihydrochalcone with bactericidal activity against Clostridioides difficile.

30. The method of claim 28, wherein the dihydrochalcone glycoside is phloridzin.

31. The method of claim 28, wherein the stilbene glycoside is polydatin.

32. The method of claim 28 or 29, wherein the dihydrochalcone is phloretin.

33. The method of claim 28 or 29, wherein the stilbene is resveratrol.

34. The method of any one of claims 27-33, wherein the subject is a human.

35. The method of any one of claims 27-34, wherein the pharmaceutical composition is administered to the subject orally.

36. The method of claim 28, wherein treatment of the Clostridioides difficile infection is effected through conversion of the stilbene glycoside or the dihydrochalcone glycoside to a stilbene or a dihydrochalcone by a microorganism in the digestive or GI tract of the subject.

37. The pharmaceutical composition of any one of claims 1-26 for use in treating a Clostridioides difficile infection.

38. A kit comprising the pharmaceutical composition of any one of claims 1-26.

39. A food product comprising:
(i) a microorganism capable of expressing an enzyme that converts a stilbene glycoside or a dihydrochalcone glycoside to a stilbene or a dihydrochalcone;
(ii) a stilbene glycoside or a dihydrochalcone glycoside, or a pharmaceutically acceptable salt thereof; and
(iii) a food.

40. A nutraceutical comprising:
(i) a microorganism capable of expressing an enzyme that converts a stilbene glycoside or a dihydrochalcone glycoside to a stilbene or a dihydrochalcone;
(ii) a stilbene glycoside or a dihydrochalcone glycoside, or a pharmaceutically acceptable salt thereof;
(iii) a food; and
(iv) a dietary supplement.
